(19) United States
(12) Patent Application Publication
(10) Pub. No.: US 2020/0397348 A1
(43) Pub. Date: Dec. 24, 2020

(54) PROCESS, COMPUTER PROGRAM AND DEVICE FOR CLASSIFYING ACTIVITIES OF A PATIENT

(71) Applicant: Drägerwerk AG & Co. KGaA, Lübeck (DE)

(72) Inventors: Frank FRANZ, Stockelsdorf (DE); Jasper DIESEL, Lübeck (DE)

(21) Appl. No.: 16/764,644

(22) PCT Filed: Nov. 13, 2018
(86) PCT No.: PCT/EP2018/081069

§ 371 Date: May 15, 2020

(30) Foreign Application Priority Data
Nov. 17, 2017 (DE) 10 2017 010 649.5

(51) Int. Cl. A61B 5/11; A61B 5/00

(52) U.S. Cl. CPC A61B 5/1128; A61B 5/1114; A61B 5/002; A61B 5/1118; A61B 5/7264; A61B 5/0013

(57) ABSTRACT

A process, a device and a computer program classify activities of a patient (100) based on image data of the patient (100) in a patient positioning device (110). The process (10) includes a detection (12) of an activity of the patient (100) based on the image data and a determination (14) of classification information for the activity from the image data. The classification information includes at least information on whether the detected activity of the patient (100) was elicited actively by the patient (100) or passively by an outside effect. The process (10) further includes a provision (16) of information on the activity and of the classification information.

### CROSS REFERENCE TO RELATED APPLICATIONS

This application is a United States National Phase Application of International Application PCT/EP2018/081069, filed Nov. 13, 2018, and claims the benefit of priority under 35 U.S.C. § 119 of German Application 10 2017 010 649.5, filed Nov. 17, 2017, the entire contents of which are incorporated herein by reference.

### TECHNICAL FIELD

Exemplary embodiments pertain to a process, to a device and to a computer program for classifying activities of a patient based on image data of the patient, especially but not exclusively on a concept for the automated detection and classification of intrinsic and extrinsic activities of the patient.

### TECHNICAL BACKGROUND

An automatic monitoring of intensive care patients is carried out according to the conventional technology primarily by monitoring vital parameters by means of medical devices (e.g., hemodynamic monitoring, ventilator) at the bedside. The medical devices shall generate an alarm when undesired physiological states develop. However, the monitoring of state variables such as of the activity of the patient is just as important.

Such monitoring has hitherto been performed mainly by attending nursing staff, which should look after the patient at very close intervals at times, depending on his status, which is in turn associated with immense costs.

A plurality of vital parameters are determined for the patients in an intensive care unit. Examples of this are the heart rate, the respiration rate or the oxygen saturation. These vital parameters make possible an improved assessment of the health status of the patient. Another vital parameter, to which markedly less attention has hitherto been paid, is the activity of the patient himself. In particular, an automatic monitoring of the patient's activity is possible only conditionally according to the current state of the art. It can be assumed in this connection that the determined patient activity is of great significance for a number of applications. Examples of this are the activity as a global vital parameter, the detection of wake-up events, the assessment of the depth of sedation or the assessment of the ability of individual body parts to move.

Most prior-art processes and devices make use of pressure sensors in contact with the patient himself or at least with his bed or of acceleration sensors, which are attached directly to the patient.

For example, pressure sensor-based systems are known, which shall be used for many different tasks. Such a system is characterized by pressure sensors, which are arranged under the mattress at a patient bed. Movements of the person lying on the mattress are recorded and analyzed. Pressure-based systems are arranged directly in the vicinity of the patient and may thus compromise cleaning and hygiene. In addition, such sensors are connected statically to the mattress of the patient and therefore they also have a static detection area.

The publication “Implementation of a Real-Time Human Movement Classifier Using a Triaxial Accelerometer for Ambulatory Monitoring” by Karantonis et al. from the year 2006 describes a system that is said to be able with an acceleration sensor attached to the hip of a patient to detect periods of activity and rest, as well as events such as walking and falling. Sensors attached directly to the patient always monitor the patient area to which they are attached.

The patent application LU90722 describes a process for monitoring a patient in a hospital bed by means of cameras. “Situation images” are recorded here, and they are then compared with one another over time in order to detect changes. If the changes exceed a threshold value, an action is triggered. Furthermore, it is explained that edges and/or contours could be detected in the situation images, as a result of which it would be possible to exclude irrelevant body parts. It is also mentioned that by detecting objects, it is possible to obtain data that describe the posture of the patient, as well as to infer movements of individual, injured body parts.

The patent application WO 02082999 pertains to the detection of convulsive attacks by means of sequences of images. A region of interest is specified for this, the image of this region of interest is divided into smaller regions, and the degree of the change over time is quantified for each region. A processor subsequently attempts to detect periodic movements in order thus to infer convulsive attacks.

Further details can be found in
Achilles, F. (2016), Patient MoCap: “Human Pose Estimation Under Blanket Occlusion for Hospital Monitoring Applications,” International Conference on Medical Image Computing and Computer-Assisted Intervention,
Barnich, O. & Van Droogenbroeck, M. (2011), ViBe: “A universal background subtraction algorithm for video sequences,” Image Processing, IEEE Transactions on, and
Girschick, R., Donahue, J., Darrell, T. & Malik, J. (2014), “Rich feature hierarchies for accurate object detection and semantic segmentation,”, CVPR (Computer Vision and Pattern Recognition).

### SUMMARY

Therefore, there is a need for developing an improved concept for the detection and classification of patient activities. This need is met by exemplary embodiments of a process, of a device and of a computer program according to the invention.

Exemplary embodiments of the present invention are based on the discovery that camera-based monitoring systems can be used for intensive care units in order to monitor an activity of a patient. For example, events can be generated in an automated manner based on detected image data of the patient, and these events are forwarded to suitable recipients via a communication network. The events can help relieve the staff members, increase the safety of the patient and lead to an improvement of care.

Exemplary embodiments provide a process, a computer program and a device for determining an activity in two-dimensional image data and/or in three-dimensional point clouds. One application is the determination of the activity of a patient (predominantly in an intensive care unit, but, e.g., also in nursing homes, etc.). Concerning the activity, distinction can be made between intrinsic (active) and extrinsic (passive) activity. Another basic idea of exemplary embodiments is to distinguish active and passive activities of a patient and generally to make a distinction concerning patient activities at least between active and passive activities. The former (actively induced) activity is characterized in that it is induced by the patient himself or is elicited. The latter passive activity can, by contrast, be attributed to external effects.

The patient is not, as a rule, completely isolated from the outside world in an intensive care unit. Nursing staff and visitors interact with the patient physically and devices can lead to movements of the patient without the patient himself being responsible for the movement. This activity of the patient, induced from the outside, may distort measurement results of alternative, non-differentiating processes, such as sensor mattresses or inertial sensor systems.

One exemplary embodiment is a process for classifying activities of a patient based on image data of the patient in a patient positioning device. The process comprises the detection of an activity of the patient based on the image data. The process comprises, moreover, the determination of classification information for the activity from the image data. The classification information comprises at least information on whether the detected activity of the patient was elicited actively by the patient or was elicited by an external effect. The process further comprises the provision of information on the activity and of the classification information. Exemplary embodiments can thus make possible a robust detection and classification of activities for persons in patient positioning devices.

In some exemplary embodiments, the process may further make provisions for the detection of the image data in the area around the patient and around the patient positioning device with a plurality of sensors. The utilization of a plurality of sensors may lead to a robust detection and classification of the patient activities.

Further, other exemplary embodiments are based on the idea of considering a region of interest within the image data. Some exemplary embodiments can therefore limit the detection of activities to regions of interest (e.g., “bed”). A region of interest can accordingly be determined in the image data, and the detection of the activities is carried out in the region of interest. The detection and the classification of the activities can therefore be limited in exemplary embodiments to certain regions of interest. Further, provisions may be made for determining an additional area, which at least partially surrounds the region of interest. The determination of the classification information can then comprise, further, the determination of whether an activity within the region of interest corresponds to an activity in the additional region.

The provision of the information on the activity may comprise an associated time stamp in some other exemplary embodiments. This time stamp may be used, for example, for documentation purposes or also for a comparison of activities in the different regions over time. The determination of the classification information may comprise in some exemplary embodiments a checking of existing activities on the basis of the time stamp. Exemplary embodiments may therefore comprise a checking for an agreement in time or an association of the activities in the regions.

The detected activity of the patient can then be classified correspondingly as being elicited actively by the patient when the detected activity within the region of interest does not correspond to any activity in the additional region. The detected activity of the patient can be classified as being elicited passively by external effect when the detected activity within the region of interest corresponds to an activity in the additional region. The region of interest and the additional region can thus make it possible to distinguish the activities at least as active and passive activities, as well as to focus the detection on a region of interest, for example, certain limbs, body regions or the entire body of the patient.

The regions can also be tracked in other exemplary embodiments. The process can accordingly comprise a tracking of the region of interest based on the image data. In other words, the region of interest and/or the additional region can be tracked adaptively to a body part or region of the patient, which body part or region is to be monitored, based on an analysis of the image data. This may also be elicited, for example, by changes in the configuration of the patient positioning device. Exemplary embodiments can thus reduce or completely eliminate a manual tracking or defocusing in the region being monitored.

In some other exemplary embodiments, the process may comprise the detection of at least one additional person in the area surrounding the patient by means of the image data and the detection of interactions between the at least one additional person and the patient. Based on the interaction, it is then possible to determine the classification information. Exemplary embodiments can thus introduce information on additional persons in the scene and on the activities of such persons into the detection and the classification of the patient activity. It is also possible in some exemplary embodiments to take into consideration the detection of a change in the configuration of the patient positioning device based on the image data or also based on other information, for example, feedbacks on the degree of adjustment of the patient positioning device, in the determination of the classification information. For example, it is also possible in some exemplary embodiments to define a plurality of regions of interest with corresponding additional regions in order thus to make a further distinction between different activities, e.g., the detection of an activity in the region of the eyes in order to detect a wake-up process, monitoring of the entire patient in the patient positioning device, monitoring of individual limbs, monitoring of body regions to detect paralyses (hemiplegia, paraplegia), etc.

It is also possible in some exemplary embodiments to detect an exercise device based on the image data. The classification information can then also be carried out on the basis of the presence of an exercise device. Possible exercise devices may be, for example, pedal exercisers, passive exercisers, a patient positioning device itself, etc. In some other exemplary embodiments, it is possible to determine an activity profile, which comprises information on the course over time of actively or passively elicited activities of the patient. Exemplary embodiments can thus provide diagnostic aids or means for assessing a recovery process on the basis of the profiles.

In other exemplary embodiments, “deep learning” processes can be used to process the image data. The determination of the classification information can then comprise a processing of the image data with “deep learning” processes. The use of deep learning may have advantages in the detection and classification of new activities.

Moreover, exemplary embodiments create a device with a computer, which is configured to carry out a process according to the above description. Exemplary embodiments also create a computer program with a program code for carrying out one of the processes according to the above description when the program code is executed on a computer, on a processor or on a programmable hardware component.

Further advantageous embodiments will be described in more detail below on the basis of the exemplary embodiments shown in the drawings, to which exemplary embodiments are not. however, generally limited as a whole. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a block diagram of an exemplary embodiment of a process for classifying activities of a patient;

FIG. 2 is a schematic top view showing a patient and a patient positioning device in an exemplary embodiment;

FIG. 3 is a flow chart of another exemplary embodiment;

FIG. 4 is a block diagram of another exemplary embodiment of a process for tracking a region of interest;

FIG. 5 is a schematic perspective view to illustrate a region of interest and another region in an exemplary embodiment;

FIG. 6 is a block diagram of another exemplary embodiment of a process for distinguishing intrinsic and extrinsic activities of a patient; and

FIG. 7 is an illustration for the further processing of activities marked as intrinsic and extrinsic in an exemplary embodiment.

### DESCRIPTION OF PREFERRED EMBODIMENTS

Referring to the drawings, different exemplary embodiments will be described now in more detail with reference to the attached drawings, in which some exemplary embodiments are shown.

Identical reference numbers can designate identical or comparable components in the following description of the attached figures, which show only some examples of exemplary embodiments. Further, summary reference numbers are used for components and objects that are present as a plurality of components and objects in an exemplary embodiment or in a drawing, but are described together in respect to one or more features. Components or objects that are described with the same reference numbers or with summary reference numbers may have identical but optionally also different configurations in respect to individual features, a plurality of features or all features, for example, their dimensioning, unless something different is explicitly or implicitly shown in the description. Optional components are represented with broken lines or arrows in the figures.

Even though exemplary embodiments may be modified and varied in different manners, exemplary embodiments are shown in the figures as examples and will be described in detail here. It should, however, be made clear that it is not intended to limit exemplary embodiments to the respective disclosed forms, but exemplary embodiments shall rather cover all functional and/or structural modifications, equivalents and alternatives, which are within the scope of the present invention. Identical reference numbers designate identical or similar elements in the entire description of the figures.

It should be noted that an element that is described as being “connected” or “coupled” with another element may be connected or coupled directly with the other element, or that elements located in between may be present. If, by contrast, an element is described as being “directly connected” or “directly coupled” with another element, no elements located in between are present. Other terms that are used to describe the relationship between elements should be interpreted in a similar manner (e.g., “between” versus “directly in between,” “adjoining” versus “directly adjoining,” etc.).

The terminology that is being used here is used only to describe certain exemplary embodiments and shall not limit the exemplary embodiments. As being used here, the singular forms “one” and “the” shall also include the plural forms, unless the context unambiguously indicates something else. Further, it should be made clear that terms, for example, “contains,” “containing,” “has,” “comprises,” “comprising” and/or “having,” as being used here, indicate the presence of said features, integers, steps, work processes, elements and/or components, but they do not exclude the presence or the addition of one or more features, integers, steps, work processes, elements, components and/or groups thereof.

Unless defined otherwise, all the terms used here (including technical and scientific terms) have the same meaning that a person having ordinary skill in the art in the area to which the exemplary embodiments belong attributes to them. It should further be made clear that expressions, e.g., those that are defined in generally used dictionaries, are to be interpreted as if they had the meaning that is consistent with their meaning in the context of the pertinent technique, and they are not to be interpreted in an idealized or excessively formal sense, unless this is expressly defined here.

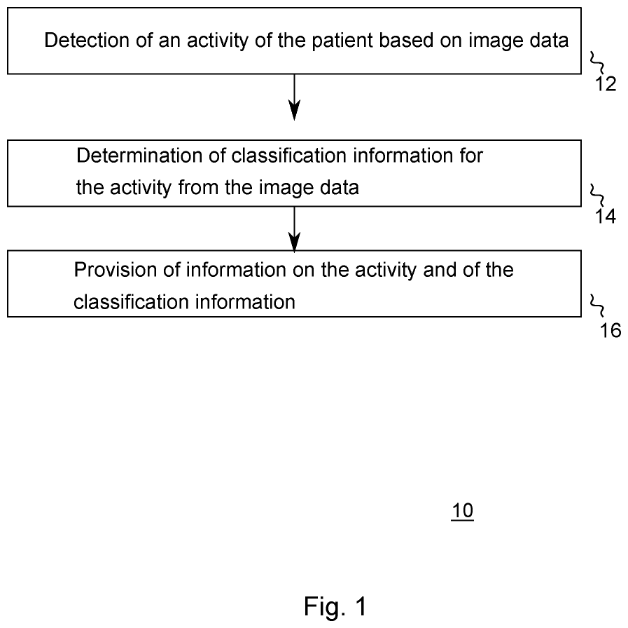

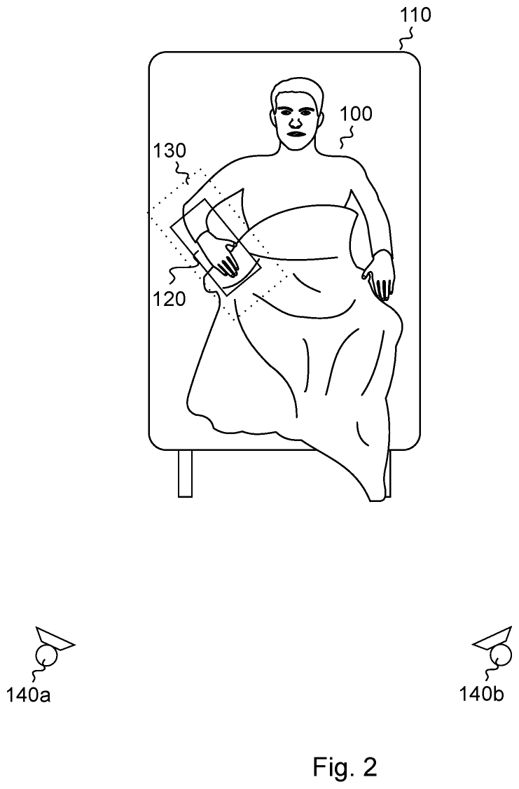

FIG. 1 shows in a block diagram an exemplary embodiment of a process 10 for classifying activities of a patient 100 based on image data of the patient 100 in a patient positioning device 110, and FIG. 2 shows the patient 100 in the patient positioning device 110. As is shown in FIG. 1, the process comprises the detection 12 of an activity of the patient based on the image data and the determination 14 of classification information for the activity from the image data, wherein the classification information contains at least information on whether the detected activity of the patient was elicited actively by the patient or passively by an external effect. The process 10 further comprises the provision 16 of information on the activity and of the classification information.

The detection of the patient activity is defined in exemplary embodiments as an at least simple determination of a piece of information on whether or not an activity or movement is present. In a simple embodiment, this information may also be simple binary information. Moreover, the classification information may also be binary information, which indicates whether a detected activity is classified or categorized as passive or active activity. The provision of the information may thus also correspond to the output of binary information.

A plurality of sensors 140a, 140b are used in some exemplary embodiments to detect the image data in the area surrounding the patient 100 and the patient positioning device 110, as this is illustrated in FIG. 2. The image data may accordingly be detected in exemplary embodiments by one image sensor or camera or a plurality of image sensors or cameras. The sensors may be two-dimensional or more than two-dimensional and also detect signals in the invisible range, e.g., infrared signals, which also make possible a corresponding processing of image data recorded in darkness. The image data themselves may also contain, for example, depth information (third dimension), which makes possible a correspondingly improved processing or robustness of the detection and classification of the activities.

Another exemplary embodiment is a computer program with a program code for executing one of the processes described here when the program code is executed on a computer, on a processor or on a programmable hardware component. Another exemplary embodiment is a device for classifying activities of a patient based on image data of the patient in a patient positioning device, with a computer for carrying out one of the processes described here. The computer may correspond in exemplary embodiments to any desired controller or processor or to a programmable hardware component. For example, the process 10 may also be implemented as software, which is programmed for a corresponding hardware component. The computer may thus be implemented as programmable hardware with correspondingly adapted software. Any desired processors, such as digital signal processors (DSPs) or graphics processors may be used. Exemplary embodiments are not limited to a certain type of processor. Any desired processors or even a plurality of processors are conceivable for the implementation of the computer.

In one exemplary embodiment, such a device may comprise 1 . . . n sensors 140a, 140b, which are oriented such that they cover at least the patient 100 and his closer surroundings, where n corresponds to a positive integer. In some exemplary embodiments, the sensors 140a, 140b may operate essentially independently from the lighting conditions provided by light visible for human beings. The image data (e.g., color images or/and infrared images or/end depth images) of the scene being monitored are then provided for a computer via the one or more sensors 140a, 140b and via a corresponding infrastructure (e.g., a network).

Different types of cameras or depth cameras may be used in exemplary embodiments. The processes being described here can be carried out with different types of cameras, as well as with one camera or with a plurality of cameras. Some exemplary embodiments use depth cameras, which possibly facilitate the definition of the regions and the distinction between intrinsic/extrinsic activities. The process also functions in exemplary embodiments, in principle, with one sensor. If a plurality of sensors are used, shadowing effects can be reduced and the process can be made more robust. An additional step is carried out in some exemplary embodiments for the extrinsic calibration of the cameras/sensors.

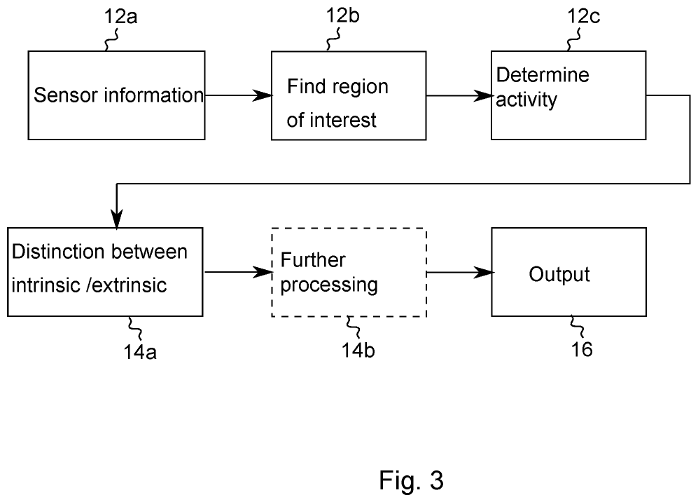

FIG. 3 shows a flow chart of another exemplary embodiment. The flow chart illustrates individual process steps in an exemplary embodiment, in which a region of interest I 120 is considered. A region of interest 120 is determined in the image data in this exemplary embodiment, and the detection of the activity is carried out in the region of interest 120. An additional region 130 is shown in FIG. 2 in addition to the region of interest 120. An additional region 130, which at least partially surrounds the region of interest, may be additionally determined in exemplary embodiments. As is shown in FIG. 3, the sensor information is detected 12a at first in the form of the image data. The region of interest 120 is then identified 12b in the pieces of sensor information. It may be, for example, a patient positioning device 110, the patient 100 or a certain region of the body of the patient 100. The activity is then determined in the region of interest 120, cf. step 12c. The activity is then distinguished 14a as being intrinsic (active) or extrinsic (passive) activity and the classification information is determined correspondingly. The output 16 is carried out after a possible or optional further processing 14b of the results obtained thus far.

The sensors operate, for example, in a contactless manner and are arranged at a distance of a few meters from the patient. Therefore, the patient is not compromised physically by the sensor system in some exemplary embodiments and the sensor system is not located in the area that is critical for hygiene. In some other exemplary embodiments of the device, the computer is implemented as a processor unit, which is connected to the 1 . . . n sensors and on which the process steps described can be carried out. Further, one or more communication connections may be implemented in exemplary embodiments in order to forward the output of the computer to receiving systems. The process steps shown in FIG. 3 will be explained in more detail below.

The process waits for the sensor information or the image data of the 1 . . . n sensors as the input. The concrete embodiment of the next steps differs slightly depending on the particular image data the sensors are delivering. If n>1 sensors are used, the input data of the sensors can be connected in space, for example, these could be combined into a three-dimensional point cloud, which will then form the image data for the further processing.

The process finds the region of interest 120 specified by the user or a second or other system in the second step 12b. Typical regions of interest 120 could be the patient positioning device (PLV) 110 with the objects located thereon. A limitation to the entire patient 100 himself or to certain body regions would be conceivable as well.

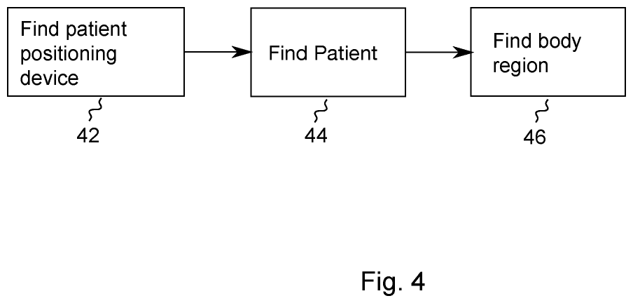

FIG. 4 shows a block diagram of another exemplary embodiment of a process for tracking a region of interest and it summarizes once again the examples mentioned here for a region of interest 120 in a flow chart. Accordingly, the patient positioning device 110 can be found at first in the image data, step 42. A patient 100 can then be identified in the area of the patient positioning device 110, step 44. A body region can then be found in the area of the patient 100 in a next step 46. Detection of a particular object in the scene may take place in some exemplary embodiments. A number of different processes that could be used are available for the object detection. Processes of deep learning, as described, for example, by Girschick et al., see above, belong to the state of the art. Object detectors, which are tailored especially to the type (or to the object) of the individual region of interest 120, as they will be explained in even more detail below, may also be used in some other exemplary embodiments.

The activity in the region of interest 120 specified in step 12b is determined in step 12c in FIG. 3. This can be accomplished in exemplary embodiments by the time series of the sensor data being considered. The partial image of the sensor data, which corresponds to the region of interest 120, can be determined now for different times (for example, every 0.1 sec), so that a chronologically meaningful sequence of partial images is formed. The activity can be quantified later by the analysis of the changes in the partial images over time. The analysis of the changes in the n partial images, which shall be designated by t_1 . . . t_n in their chronological sequence, may be carried out in different manners.

Various possibilities may be considered for this in exemplary embodiments. One possibility is the determination of an absolute differential image. For example, the absolute difference of the pixel values can be determined for this for each pixel position, for example, for each pair t_i and t_(i−1), which leads to a resulting differential image D as a result. The individual differences can then be compared with a threshold value s_1 and

$$V ( x , y ) = \{ \begin{matrix}0 , if D ( x , y ) < {s}_{1} \\ 1 , if D ( x , y ) \geq {s}_{1}\end{matrix}$$

can be written correspondingly. The sum of the pixels in V with the value 1 can be used now as an indicator of the activity at the time i.

Another possibility for the detection of an activity is a scanned value-based subtraction of the background information (cf. English “sample based background subtraction”). Depending on the selection of the needed threshold values in the preceding concept, the corresponding indicators may be susceptible to noise or may not be sensitive enough to real movement. At least some exemplary embodiments may therefore use methods that take the history of a pixel into account in more detail in order to decide whether or not this pixel represents activity. A known and successful example for this is ViBe, Barnich, O., et al., see above. ViBe generates, in turn, an activity map, in which a pixel contains the value 1 when it experiences activity, and the value 0 when this is not the case. The sum of the pixel values is an indicator of the activity here as well.

The parameters for activity may, moreover, also be standardized by the sums being divided by the number of pixels of the partial image being considered. The above-described processes yield a chronologically meaningful sequence (time series) of parameters k_1 . . . k which describe the activity at the time t_1 . . . t_m.

Further, exemplary embodiments make a distinction at least between intrinsic or active activity when the activity is elicited by the patient himself, and extrinsic or passive activity when the activity is categorized as being elicited from the outside. As was explained above, even though the measured or detected activity can be limited in step 12c to a defined region, the origin of the activity is nevertheless unknown to the system so far. The determination 14 of the classification information is therefore carried out.

The process makes a distinction in this step 14 between intrinsic and extrinsic activity. Other categorizations may also be made in other exemplary embodiments. It may be assumed, in general, that activity also occurs in the case of extrinsic activity in the closer surroundings of the region of interest 120 being considered. This idea of detecting potential extrinsic activity is pursued in this exemplary embodiment in interaction with the additional region 130, cf. FIG. 2. Further, optionally more differentiated possible solutions will be explained below.

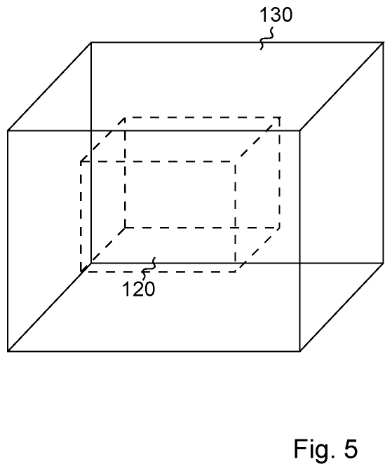

The process determines now in this exemplary embodiment an additional region Z 130 around the region of interest I 120 proper. Z could be, for example, a box, which corresponds to a box I enlarged by a value W in each dimension minus the box 1 itself. W could also depend on the size proper of I. An example for the regions I and Z is shown in FIG. 5. FIG. 5 shows an illustration to illustrate a region of interest 120, shown here as a box, and an additional region 130, likewise shown as a box, in an exemplary embodiment.

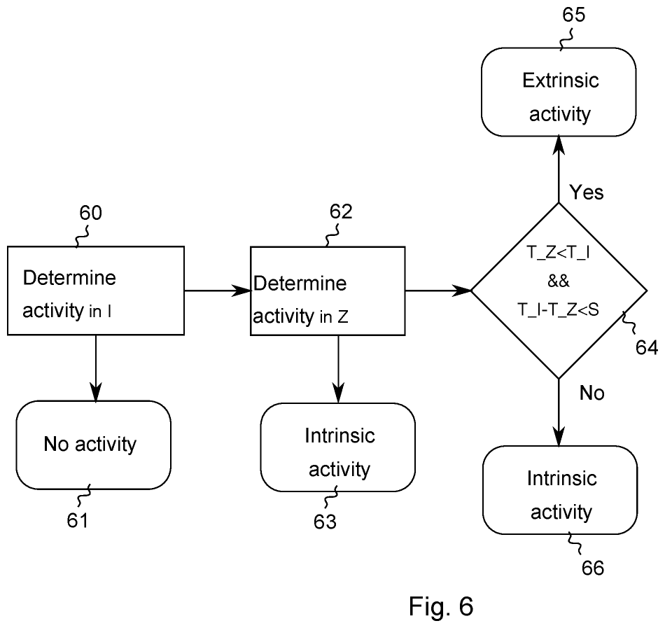

If the region Z 130 has been specified, the distinction between extrinsic and intrinsic activity can be carried out in the region of interest 120 I. FIG. 6 shows a block diagram of another exemplary embodiment of a process for making a distinction between an intrinsic and extrinsic activity of a patient 100. As was explained above, activity in I is determined at first in a step 60. If no activity is found here, this is also the current output of the process, as this is shown in step 61. Otherwise, the determined activity A_1 as well as the corresponding time stamp T_1 are made available. The provision 16 of the information on the activity also comprises in this exemplary embodiment the provision of a corresponding or associated time stamp. The process then continues by the previous step “determine activity” for the region Z being repeated in step 62. If no activity is found now in Z, the activity in I can be outputted as activity marked as intrinsic in step 63.

If an activity is detected in Z, a comparison of the time stamps follows in step 64, T_Z being the time stamp belonging to the detected activity in region Z. The determination 14 of the classification information thus comprises in this exemplary embodiment a checking of existing activities on the basis of the time stamp. If the process consequently detects activity in Z before an activity in I (T_Z<T_I in step 64), which is not, furthermore, older than a parameter S, as the activity in I (T_I−T_Z<S in step 64), the process assumes that the activity is an extrinsic activity, and it outputs this in step 65. If, however, the activity in I is detected before or simultaneously with activity in Z, this is classified as intrinsic activity and is outputted in step 66. It is correspondingly determined in some exemplary embodiments whether an activity within the region of interest I 120 corresponds to an activity in the additional region Z 130. The detected activity of the patient 100 can then be classified as being activity elicited by the patient when the detected activity within the region of interest 120 does not correspond to any activity in the additional region 130. Analogously, the detected activity of the patient 100 can be classified as being elicited passively by external effect when the detected activity within the region of interest I 120 corresponds to an activity in the additional region Z 130.

In other words, the activity in the region of interest I 120 can be classified as being elicited actively by the patient 100 when no (corresponding) activity is detected in the additional region Z 130. The activity in the region of interest I 120 can be classified as being elicited passively from the outside if a (corresponding) activity in the additional region is detected within a time period S before or simultaneously with the activity in the region of interest I 120. Further processing of the data obtained hitherto, i.e., of the information on the activity and the classification information, is optional and will be explained in more detail below. The process yields in the output or the provision 16 the data obtained in the previous steps, for example, to a receiving system. This may take place, e.g., via a communication connection, for example, Ethernet.

The process described above can be made more robust to disturbances in some other exemplary embodiments, for example, by an automatic tracking of the region of interest I 120 (English tracking) and by the use of more sensors, which was already described above. The region of interest I 120 can be found automatically and tracked in a scene in some exemplary embodiments. The process 10 further comprises in this exemplary embodiment a tracking of the region of interest 120 based on the image data. A user interaction can thus be reduced or avoided in such exemplary embodiments if the region of interest I 120 is shifted in the scene. For example, it is also possible to use specially tailored detectors for different regions of interest. For the detection of a patient positioning device 110 with image processing means, reference is made, for example, to the document DE 10 2017 006529.2, which discloses a related concept. Reference is made, for example, to Achilles et al., see above, concerning the determination of the patient 100 per se or of individual body parts of the patient 100.

The distinction between intrinsic and extrinsic activity can be improved in other exemplary embodiments; for example, the cause of an extrinsic activity can also be determined. “Activating objects,” e.g., persons or devices, which are introduced into the area surrounding the region of interest proper, can be detected now. In some exemplary embodiments, the process 10 comprises the detection of at least one additional person in the area around the patient by means of the image data and the detection of interactions between the at least one additional person and the patient 100. Further, the classification information can be determined now based on the interaction. Manipulations by additional persons may occur in the scenarios being considered. An extrinsic movement or action of the patient 100 frequently takes place due to manipulation by another person. This manipulation can be detected, cf. DE 102017006529.2.

Movement of the bed can be detected in other exemplary embodiments; for example, the patient positioning device 110 may be adjusted. This may possibly also happen in some exemplary embodiments from a remote location, without persons being present in the vicinity. It would be possible to determine whether this happens with a process from the documents DE 102015013031 or DE 3436444. A process, with which the configuration of a patient positioning device can be determined, is described there. If the process determines a change in the configuration during the time period in question, activity occurring during this time period can be marked or detected as being extrinsic (due to a change in the configuration of the patient positioning device). The process 10 may also comprise now the detection of a change in the configuration of the patient positioning device based on the image data and the determination of the classification information based on the change in the configuration.

A plurality of regions of interest with corresponding additional regions may, in general, also be defined in exemplary embodiments. Since the process acts based on image data, a plurality of pairings of regions of interest and other regions may be analyzed in parallel or also serially. It would thus also be possible, for example, to monitor halves of the body of a patient 100 separately in order thus to detect hemiplegia and paraplegia.

Exercise devices, such as pedal exercisers and passive exercisers, are said to be helpful in mobilizing patients. Exercise devices, which have been introduced into the area surrounding the region of interest I 120, can also be detected by means of object detection and tracking processes. Depending on the device, the device also may or may not be provided with a motor. The movement of the patient 100 can be supported or even performed completely in the first case. In the second case, it is used at least to encourage the patient to move. However, the movement is motivated in any case not only intrinsically, so that a separate marking of the activity may preferably be carried out by the process for the period during which an exercise device was detected in the region of interest. Exemplary embodiments may therefore make provisions for the detection of an exercise device based on the image data, and for the determination 14 of the classification information based on the presence of an exercise device.

It is also possible to include an activity of the patient from the past to make it possible to distinguish the type of the exercise device. Some exemplary embodiments may establish and store the history of the activities of a patient. If, for example, a patient 100 just had a high intrinsic activity, it is probable that the exercise device shall only be used for support. If the patient had no intrinsic activity, a fully supporting system is very likely.

Moreover, some exemplary embodiments may also determine one or more activity profiles, which comprise information on changes over time in actively or passively elicited activities of the patient. The process can thus be refined such that the information obtained on the intrinsic and extrinsic activity will be subjected to further processing. This could happen either directly on the processor unit/computer of the device described, and thus also before the output (to additional systems), or, if possible, also in another system, which is receiving the data obtained so far. One possibility of further processing would be the removal of extrinsic activity. This could be done by removing areas with extrinsic activity from the activity data simply without replacement. If the activity is averaged now over a time window, an indicator is obtained for the intrinsic activity of the object, for example, the patient.

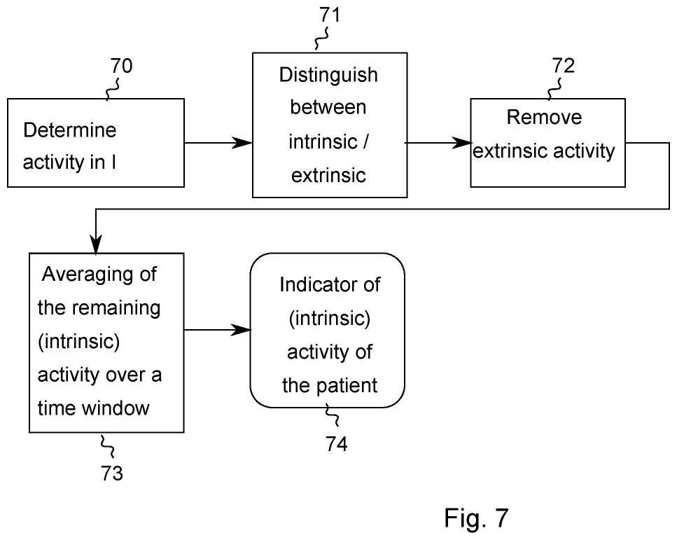

FIG. 7 shows an illustration for the further processing of activities marked as intrinsic and extrinsic in an exemplary embodiment. The flow chart shown illustrates the further processing of the activities marked as intrinsic and extrinsic, so that an indicator is available at the end of the process for the intrinsic activity of the patient. An activity in the region of interest is determined at first in step 70, and distinction is subsequently made between intrinsic and extrinsic activity in step 71. The activity categorized as extrinsic is removed in step 72 in the exemplary embodiment shown and the remaining intrinsic activity is averaged over a time window in step 73. An indicator can then be outputted as a result for the intrinsic activity of the patient.

The other way around, time periods with intrinsic activity can also be removed in other exemplary embodiments in the third step 72 of the flow chart, as a result of which an indicator can be obtained for the activity elicited from the outside (extrinsic activity). The signal could also be smoothened in order to compensate errors of measurement. This could be accomplished, for example, with a “moving average” filter. An average could similarly be determined over a longer time period and used as an output.

The data could also be compared with a threshold value and an event could be used as an output if the current value is above or below the threshold value.

Furthermore, the data obtained until the current time X could be extrapolated. It would thus be possible, for example, to predict a value exceeding a threshold value, before this value would actually appear. It would also be possible to perform a frequency analysis on the activity signal in order to find out whether the movement occurs at one frequency or at a plurality of frequencies and what these frequencies are.

In other exemplary embodiments, the device described up to now could provide a number of different outputs, which may be meaningful for different applications, with the described process or with the process itself. For example, the calculated activity value can be outputted as a continuous measured variable. This activity value could thus be made available as an additional vital parameter for a patient (especially if the extrinsic activity is removed). It would also be possible to make available only the extrinsic activity, in which case it would be possible to monitor how often and for how long a patient was disturbed during certain time periods (cf. DE 102017006529.2).

The intrinsic activity value (especially combined with a threshold value comparison) can be used to detect wake-up situations when threshold values are exceeded. This is meaningful, for example, when a patient is arriving from an operating room and it is necessary to wait for him to wake up. Rhythmic movements can likewise be detected in some exemplary embodiments. If a frequency analysis shows, for example, that the movement is subject to periodic changes, the determined frequency can be used to detect (tonic clonic) seizures (cf. WO 02082999). If a single-time, intense movement is detected, there is a possibility that a supported body part has dropped off, which can thus be indicated in some exemplary embodiments. If a half of the body along the sagittal plane or along the transverse plane is defined as the region of interest, the development of a hemiplegia can be followed up in some exemplary embodiments in conjunction with the intrinsic activity, and the development of a paraplegia can be followed up in the latter case. Further, a body part or a body region could be defined as a region of interest in other exemplary embodiments and the recovery/changes in that region could be monitored accordingly.

Exemplary embodiments can analyze, in general, image data of patients, which are detected by means of cameras, and the patients can thus be observed. The status of the patient (convulsion) can be inferred by means of detected activity/movement. Distinction can be made between active and passive activity and activity induced from the outside can thus be detected and distinguished. Exemplary embodiments are not limited to the detection of certain activities such as convulsions and they are thus suitable for the detection of other types of activities as well. In addition, some exemplary embodiments can track a region of interest adaptively or automatically and thus observe the recovery of a body part. Exemplary embodiments are therefore robust in respect to shadowing and moving regions of interest. Exemplary embodiments make do without sensors attached directly to the patient. In particular, the focus can thus be placed on special regions of interest, which may possibly change over time.

The “deep learning” model is used in another exemplary embodiment to process the image data. For example, the model infers from an image sequence as an input directly whether an activity of the patient is intrinsic or extrinsic right now. An algorithm or a process would implicitly learn in such an exemplary embodiment that, e.g., a manipulation of the patient by another person is causing extrinsic activity. An input of an image sequence would be followed, for example, by a video portion classification by a deep neuronal network in order to determine the classification information.

The features disclosed in the above description, in the claims and in the drawings may be of significance for the implementation of exemplary embodiments in their different configurations both individually and in any combination and, unless something else appears from the description, they may be combined with one another as desired.

Even though some aspects were described in connection with a process or with a device, it is obvious that these aspects also represent a description of the corresponding device and of the corresponding process, so that a block or an element of a device can also be considered to be a corresponding process step or as a feature of a process step and vice versa. Analogously to this, aspects that were described in connection with or as a process step also represent a description of a corresponding block or detail or feature of a corresponding device.

Depending on certain implementation requirements, exemplary embodiments of the present invention may be implemented in hardware or in software. The implementation may be carried out with the use of a digital storage medium, for example, a floppy disk, a DVD, a Blue-Ray disk, a CD, a ROM, a PROM, an EPROM, an EEPROM or a FLASH memory, a hard drive or another magnetic or optical memory, on which electronically readable control signals are stored, which can or do interact with a programmable hardware component such that the particular process is carried out.

A programmable hardware component may be formed by a processor, a computer processor (CPU=Central Processing Unit), a graphics processor (GPU=Graphics Processing Unit), a computer, a computer system, an application-specific integrated circuit (ASIC=Application-Specific Integrated Circuit), an integrated circuit (IC=Integrated Circuit), a System on Chip (SOC=System on Chip), a programmable logic element or a field-programmable gate array with a microprocessor (FPGA=Field Programmable Gate Array).

The digital storage medium may therefore be machine- or computer-readable. Some exemplary embodiments consequently comprise a data storage medium, which has electronically readable control signals, which are capable of interacting with a programmable computer system or with a programmable hardware component such that one of the processes described here is carried out. An exemplary embodiment is thus a data storage medium (or a digital storage medium or a computer-readable medium), on which the program for executing one of the processes described here is recorded.

Exemplary embodiments of the present invention may generally be implemented as program, firmware, computer program or computer program product with a program code or as data, wherein the program code or the data act such as to carry out a process when the program is running on a processor or on a programmable hardware component. The program code or the data may also be stored, for example, on a machine-readable medium or data storage medium. The program code or the data may also be, among others, in the form of a source code, machine code or byte code as well as another intermediate code.

Another exemplary embodiment is, furthermore, a data stream, a signal sequence or a sequence of signals, which data stream, signal sequence or signal sequence represents the program for carrying out a process being described here. The data stream, the signal sequence or the sequence of signals may be configured, for example, such as to be transferred via a data communication connection, for example, via the Internet or another network. Exemplary embodiments are thus also signal sequences representing data, which signal sequences are suitable for transmission via a network or a data communication connection, wherein the data represent the program.

A program according to an exemplary embodiment may implement one of the processes during its execution, for example, by the program reading storage locations or writing a datum or a plurality of data into these storage locations, as a result of which switching operations or other operations are elicited in transistor structures, in amplifier structures or in other electrical, optical, magnetic components or in components operating according to another principle of function. Data, values, sensor values or other pieces of information can correspondingly be detected, determined or measured by reading a storage location. A program may therefore detect variables, values, measured variables and other pieces of information by reading from one or more storage locations, as well as bring about, prompt or perform an action as well as actuate other devices, machines and components by writing into one or more storage locations.

The above-described exemplary embodiments represent only an illustration of the principles of the present invention. It is obvious that modifications and variations of the devices and details described here will be clear to other persons skilled in the art. It is therefore intended that the present invention shall be limited only by the scope of protection of the following patent claims and not by the specific details, which were presented here on the basis of the description and the explanation of the exemplary embodiments.

While specific embodiments of the invention have been shown and described in detail to illustrate the application of the principles of the invention, it will be understood that the invention may be embodied otherwise without departing from such principles.

1. A process for classifying activities of a patient based on image data of the patient in a patient positioning device, the process comprising the steps of:
detecting an activity of the patient based on the image data;
determining classification information for the activity from the image data, wherein the classification information comprises at least information on whether the detected activity of the patient was elicited actively by the patient or passively by an outside effect; and
providing information on the activity and of the classification information.

2. A process in accordance with claim 1, further comprising the step of detecting an area surrounding the patient and the patient positioning device with a plurality of sensors to provide the image data.

3. A process in accordance with claim 1, further comprising the step of determining a region of interest in the image data, wherein the detection of the activity is carried out in the region of interest.

4. A process in accordance with claim 3, further comprising the step of determining an additional region, which at least partially surrounds the region of interest.

5. A process in accordance with claim 4, wherein the determination of the classification information further comprises the determination of whether an activity within the region of interest corresponds to an activity in the additional region.

6. A process in accordance with claim 5, wherein the detected activity of the patient is classified as having been elicited actively by the patient if the detected activity within the region of interest does not correspond to an activity in the additional region.

7. A process in accordance with claim 5, wherein the detected activity of the patient is classified as having been elicited passively by an outside effect if the detected activity within the region of interest corresponds to an activity in the additional region.

8. A process in accordance with claim 1, wherein the provision of the information on the activity comprises providing an associated time stamp.

9. A process in accordance with claim 8, wherein the determination of the classification information comprises a checking of existing activities on the basis of the time stamp.

10. A process in accordance with claim 3, further comprising tracking the region of interest based on the image data.

11. A process in accordance with claim 1, further comprising the steps of:
detecting at least one additional person in the area around the patient based on the image data;
detecting interactions between the detected at least one additional person and the patient; and
determining the classification information based on the interaction.

12. A process in accordance with claim 1, further comprising the steps of:
detecting a change in the configuration of the patient positioning device based on the image data; and
determining the classification information based on the change in the configuration.

13. A process in accordance with claim 1, further comprising the steps of:
detecting an exercise device based on the image data; and
determining the classification information based on the presence of an exercise device.

14. A process in accordance with claim 1, further comprising the step of providing an activity profile, which comprises information on the course over time of actively or passively elicited activities of the patient.

15. (canceled)

16. A process according to claim 1, wherein a computer program is provided with a program code for executing the processes when the program code is run on a computer, on a processor or on a programmable hardware component.

17. A device for classifying activities of a patient based on image data of a patient in a patient positioning device, the device comprising:
a computer configured to:
receive image data and detect an activity of the patient based on the image data;
determine classification information for the activity from the image data, wherein the classification information comprises at least information on whether the detected activity of the patient was elicited actively by the patient or passively by an outside effect; and
provide information on the activity and of the classification information.

18. A device for classifying activities of a patient in accordance with claim 17, further comprising a plurality of sensors to provide the image data, wherein:
the sensors detect an area surrounding the patient and the patient positioning device;
and output the image data to the computer.

19. A device for classifying activities of a patient in accordance with claim 18, wherein the computer is further configured to:
determine a region of interest in the image data, wherein the detection of the activity is carried out in the region of interest;
determine an additional region, which at least partially surrounds the region of interest; and
to determine whether an activity within the region of interest corresponds to an activity in the additional region, wherein:
the detected activity of the patient is classified as having been elicited actively by the patient if the detected activity within the region of interest does not correspond to an activity in the additional region; and
the detected activity of the patient is classified as having been elicited passively by an outside effect if the detected activity within the region of interest corresponds to an activity in the additional region.

20. A device for classifying activities of a patient in accordance with claim 18, wherein:
the provision of the information on the activity comprises the computer providing or generating an associated time stamp;
the determination of the classification information comprises the computer checking existing activities on the basis of the time stamp.

21. A device for classifying activities of a patient in accordance with claim 18, wherein the computer is further configured to:
detect at least one additional person in the area around the patient based on the image data;
detect interactions between the detected at least one additional person and the patient; and
determine the classification information based on the interaction.
